(19) United States
(12) Patent Application Publication
(10) Pub. No.: US 2025/0144665 A1
(43) Pub. Date: May 8, 2025

(54) METHODS OF PREPARING COATINGS AND RELATED DEVICES AND SYSTEMS

(71) Applicant: THERADEP TECHNOLOGIES, INC., San Jose, CA (US)

(72) Inventors: Denis O'SULLIVAN, Dungarvan, Waterford (IE); Liam O'NEILL, Midleton, Cork (IE); Chloe FREWEN, Aherlow, Tipperary (IE)

(73) Assignee: THERADEP TECHNOLOGIES, INC., San Jose, CA (US)

(21) Appl. No.: 18/836,955

(22) PCT Filed: Feb. 8, 2023
(86) PCT No.: PCT/US2023/062219

§ 371 Date: Aug. 8, 2024

Related U.S. Application Data
(60) Provisional application No. 63/267,728, filed on Feb. 9, 2022.

(51) Int. Cl. B05D 1/08; B05D 3/10

(52) U.S. Cl. CPC B05D 1/08; B05D 3/10

(57) ABSTRACT

Coated substrates and methods of preparing such coated substrates are described. The method may include introducing an aerosol into a non-thermal plasma, wherein the aerosol comprises at least one organic compound and a first solvent. depositing the at least one organic compound on a surface of a substrate positioned downstream of the plasma to form a coating on the surface. and spraying a second solvent in aerosol form on the coating. the second solvent being the same or different from the first solvent. Spraying the second solvent may increase uniformity of the coating.

### CROSS-REFERENCE TO RELATED APPLICATIONS

This application claims priority to U.S. Provisional Application No. 63/267,728 filed on Feb. 9, 2022, which is incorporated by reference herein in its entirety.

### TECHNICAL FIELD

The present disclosure is generally directed to systems and methods for preparing coatings, and substrates such as medical devices comprising such coatings.

### BACKGROUND

In general, there are two plasma types, namely thermal equilibrium and non-isothermal equilibrium plasmas, each presenting their own challenges in adherence to substrate. Thermal equilibrium plasmas, typically hot with temperatures of approximately 10,000 Kelvin, are used in industry as plasma torches, jets, and arcs for welding. These systems are also used in thermal spray coating, often for depositing metallic and ceramic coatings onto metal surfaces for diverse applications, ranging from producing biocompatible hydroxyapatite coatings on medical implants to the deposition of protective coatings on gas turbine components. Despite the widespread use of thermal plasmas, their applications are limited by the requisite high thermal energy that prevents these devices from use in depositing temperature sensitive materials, such as proteins, polysaccharides, and other chemical and biomaterials.

In contrast, non-thermal plasmas are generally cool and can be employed in manufacturing processes including surface cleaning (including, e.g., removal of unwanted substances such as contaminants), etching (e.g., removal of bulk substrate material), activation (e.g., changing surface energies) and deposition of functional thin film coatings onto surfaces. Historically, these coating devices were limited to vacuum conditions and used only gas phase monomers to produce coatings. As a result, the chemistry of the deposited materials was inherently simplistic and these devices were not compatible with large, high molecular weight macromolecules. These plasma systems have been widely used to modify surfaces to allow for subsequent attachment of pharmaceutical agent or biomolecule through traditional wet chemistry techniques in multi-step processes, but require complex linkers and binder chemicals to prepare the surface in advance of introducing the target biological molecule.

Recent years have seen the development of plasma devices that operate at atmospheric pressure and which can also produce functional coatings using gas phase or liquid phase monomers. The switch from vacuum systems to ambient pressure allows for the use of pharmaceutical agents or biomolecules other than gas phase monomers in the production of thin films. Often, these films produce cross-linking reactions between the monomer molecules and form a cured or cross-linked polymeric coating, which then retains any non-uniformity established when the coating was produced. Other produced coatings contain active agents, but these agents lack the functionality to readily undergo reactions within the plasma. Instead, the actives are physically encapsulated by the polymeric coating formed by the reactive polymer-forming materials. Even in coatings that retain biological functionality, the pharmaceutical agent or biomolecules are cross-linked or coagulated by the plasma.

In prior systems used to deposit coatings, the liquid interacts with the plasma and then subsequently lands on the target substrate and forms the coating. The plasma may continue to act upon the deposited materials to further induce reactions or the plasma may be kept at a distance to prevent such interactions. As the plasma-liquid interaction produces immediate reactions, this means that the chemical reaction begins to occur in the gas phase as soon as the liquid droplet contacts the plasma. These reactions can continue on the surface after the droplet impacts the substrate and also contacts other droplets resulting in bonding to adjacent droplets and to the substrate, giving rise to unusual morphologies and unwanted non-uniformities. Such non-uniformities in the coating include circular structures which may relate back to the original droplets and their initial reaction in the plasma. This non-uniformity is often only visible under high optical magnification, occurring at the micron or even the nanometer level, but it can significantly impair the functionality of the final application, especially when the coating works at a cellular level.

### SUMMARY

The present disclosure includes methods of preparing substrates by depositing a coating with increased uniformity. For example, the present disclosure includes a method for preparing a substrate, the method comprising introducing an aerosol into a non-thermal plasma, wherein the aerosol comprises at least one organic compound and a first solvent: depositing the at least one organic compound on a surface of a substrate positioned downstream of the plasma to form a coating on the surface, and spraying a second solvent in aerosol form on the coating, the second solvent being the same or different from the first solvent: wherein the spraying the second solvent increases uniformity of the coating. The second solvent may be sprayed on the coating within 1 hour to 24 hours of forming the coating, for example, by depositing the at least one organic compound on the surface of the substrate. The second solvent may comprise an alcohol such as ethanol, methanol, propanol, or butanol, or another solvent such as N,N-dimethylacetimide or cyclohexanone. The second solvent may be sprayed on the coating while the coating is wet or while the coating is dry.

The at least one organic compound may comprise a pharmaceutical agent or a biomolecule, for example, wherein the coating may retains a pharmaceutical activity or biological activity of the at least one organic compound. In another example, the at least one organic compound may comprise a polymer, with or without pharmaceutical or biological activity. The organic compound may comprise at least one phosphorylcholine group, e.g., a polymer comprising one or more phosphorylcholine groups. In some examples, the aerosol introduced into the non-thermal plasma consists of or consists essentially of a pharmaceutical agent and the first solvent, or wherein the aerosol introduced into the non-thermal plasma consists of or consists essentially of a biomolecule and the first solvent. The amount of second solvent sprayed on the coating may be less than or equal to 1 ml/cm², such as less than or equal to 100 μL/cm² of the second solvent. For example, less than or equal to 10 μL/cm² of the second solvent may be sprayed on the coating. In some examples, a total volume of the second solvent applied to the substrate surface does not exceed a total volume of the first solvent applied to the substrate surface. The coating may have a thickness less than or equal to 500 nm, such as about 10 nm to about 100 nm, or about 20 nm to about 50 nm. The substrate may comprise glass, a plastic or other polymer, a metal, a metal alloy, or a ceramic, for example. According to some aspects of the present disclosure, the substrate comprises a medical device. For example, the substrate may be or comprise a medical device chosen from a microplate, a petri dish, a cell culture flask, a tube, or a cuvette.

In the methods discussed above and elsewhere herein, the at least one organic compound may be dissolved in the first solvent for at least 24 hours, at least 36 hours, or at least 48 hours prior to introducing the aerosol into the non-thermal plasma. In some aspects of the present disclosure, the method includes preparing a solution comprising the at least one organic compound and the first solvent, and introducing the solution into a nebulizer to form the aerosol at least 24 hours, at least 36 hours, or at least 48 hours after preparing the solution. In some examples herein, the second solvent is not introduced into the non-thermal plasma before spraying the second solvent on the coating (e.g., the second solvent is sprayed on the coating in the absence of a plasma). Additionally or alternatively, the substrate may be pretreated. For example, the method may further comprise pretreating the surface of the substrate before depositing the at least one organic compound on the surface. Pretreating the surface of the substrate may include, for example, plasma activation at reduced pressure (e.g., vacuum pressure), plasma activation at atmospheric pressure, flame treatment, or chemical oxidation.

In some aspects of the present disclosure, the second solvent is sprayed on the coating with an organic compound that may be the same or different than the organic compound of the coating. For example, the organic compound introduced with the first solvent into the non-thermal plasma in aerosol form may be a first organic compound, and the second solvent may be sprayed on the coating as an aerosol that comprises the second solvent and a second organic compound that is the same or different than the first organic compound.

Increasing uniformity of the coating may include decreasing or eliminating one or more defects of the coating. For example, after depositing the organic compound(s) on the substrate surface to form the coating, the coating may include one or more defects, and spraying the second solvent may decrease or eliminate the one or more defects. The defect(s) of the coating may include, for example, surface roughness, a scratch, a crack, a ridge, a depression, density variation, phase change, wrinkling, chipping, blistering, bubbling, running, staining, water marking, streaking, hazing, uneven coating, and/or pinhole formation.

The present disclosure also includes substrates prepared according to the methods discussed above and elsewhere herein. For example, the substrate prepared according to the methods discussed above may be a medical device, wherein the coating is a uniform coating having a thickness less than or equal to 500 nm, such as a thickness ranging from about 20 nm to about 50 nm. The medical device may be or comprise a microplate, a petri dish, a cell culture flask, a tube, or a cuvette, for example. In some examples, the coating comprises phosphorylcholine. The coating of the substrates prepared as disclosed herein may inhibit or prevents cell adhesion to the substrate.

### BRIEF DESCRIPTION OF THE FIGURES

The accompanying figures, which are incorporated in and constitute a part of this application, illustrates certain features of the present disclosure, and together with the description, serves to explain the principles of the present disclosure. Those of ordinary skill in the art will readily recognize that the features of a particular aspect or embodiment may be used in conjunction with the features of any or all of the other aspects or embodiments described in this disclosure.

FIGS. 1 and 2 show optical microscope images of a substrate with an organic coating before and after a solvent spraying step according to the present disclosure, as discussed in Example 1.

### DETAILED DESCRIPTION

The present disclosure includes systems, devices, and methods for preparing coated substrates, e.g., using a non-thermal plasma for deposition of an organic compound, such as a polymer, a monomer capable of polymerizing, and/or an active agent, on a substrate to form the coating. The methods herein may provide coatings that are more uniform, e.g., having fewer discontinuities across the surface, than prior coatings. The coatings and coated substrates may have industrial application in the formation of non-fouling surfaces, antimicrobial devices, and cell culture processes, among other uses.

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ±5% of a specified amount or value. All ranges are understood to include endpoints, e.g., a thickness between 20 nm and 80 nm includes a thickness of 20 nm, 80 nm, and all values between.

The methods herein may use a system comprising one or more devices for generating an aerosol (e.g., atomizer, sprayer, or nebulizer) and a plasma device including electrodes to generate a non-thermal plasma and a plasma chamber. The method may comprise introducing an aerosol comprising an organic compound into a plasma and depositing the organic compound onto a substrate to form a coating, then spraying a solvent on the deposited coating. e.g., to remove non-uniformity in the coating. The organic compound may be activated by the plasma, which may contribute to increased adhesion of the coating to the substrate. The solvent may be selected for compatibility with the organic compound of the coating. As described below, the methods herein may include an optional step of pre-treating the substrate.

The coatings herein may be prepared from one or more organic compounds, including polymers, monomers capable of polymerizing, and/or active agents having pharmaceutical and/or biological activity. The method may comprise dissolving at least one organic compound in a suitable volatile solvent to form a liquid, and generating an aerosol from the liquid, which is then introduced into a non-thermal plasma. For example, the aerosol comprising the organic molecule(s) may be sprayed or otherwise introduced into the afterglow region of a non-thermal plasma. For aerosols comprising a monomer and/or polymer, exposure to the plasma may cause the monomer and/or polymer to polymerize or further polymerize when deposited on the substrate surface to form the coating. The organic compound(s) may be deposited to form the coating in the absence of crosslinkers, linkers, or binders. In some aspects of the present disclosure, for example, the solution comprising the organic compound(s) and the solvent does not include any crosslinkers, linkers, or binders.

Organic compounds useful for the coatings herein include active agents such as pharmaceutical agents and biomolecules, wherein the coatings may retain partial or complete pharmaceutical or biological activity of the respective active agent. For example, the active agent(s) may comprise one or more antiseptics and/or antimicrobials (including antibiotics and bacteriostatic agents), biopolymers, synthetic biodegradable polymers, and combinations thereof. Exemplary organic compounds include, but are not limited to, collagen, fibrin, elastin, fibronectin, hyaluronan, chitosan, alginates, cellulose, phosphorylcholine, polypeptides, polyglycans, hormones, lipids, interferons, cartilage, recombinant blood cells, synthetic derived blood cells, platelet-rich plasma, cells (autologous and donor cells), melanocytes, stem cells, antibodies (including monoclonal antibodies), amniotic membrane materials, bovine serum albumin, proteins, clotting factors, growth factors, cytokines, chemotherapy agents, anti-inflammatory drugs, immune-suppressants, analgesics, blood pressure medications, antithrombotics, anticoagulants, antiplatelet agents, thrombolytics, antiproliferatives, antimitotic, agents that inhibit restenosis, smooth muscle cell inhibitors, fibrinolytic agents, immunosuppressive agents, vaccines, and combinations thereof. In at least one example, the aerosol comprises collagen, blood plasma, chitosan, or a combination thereof.

Organic compounds suitable for the coatings herein also include polymers, microspheres, liposomes, micelles and nanoparticles For example, the organic compounds herein may comprise polymers and monomers capable of polymerizing by exposure to the plasma. Exemplary monomers include, but are not limited to, carboxylates, methacrylates (including, e.g., methyl methacrylate, ethyl methacrylate, propyl methacrylate, butyl methacrylate, glycidyl methacrylate, trimethoxysilyl propyl methacrylate, allyl methacrylate, hydroxyethyl methacrylate, hydroxypropyl methacrylate, dialkylaminoalkyl methacrylate, fluoroalkyl methacrylate, and other alkyl methacrylates and organofunctional methacrylates), acrylates (including, e.g., methyl acrylate, ethyl acrylate, propyl acrylate, butyl acrylate, glycidyl acrylate, trimethoxysilyl propyl acrylate, allyl acrylate, hydroxyethyl acrylate, hydroxypropyl acrylate, dialkylaminoalkyl acrylate, fluoroalkyl acrylate such as heptadecafluorodecyl acrylate, and other alkyl acrylates and organofunctional acrylates), styrenes (including, e.g., α-methylstyrene), methacrylonitriles, alkenes and dienes (including, e.g., halogenated alkenes such as vinyl halides, e.g., vinyl chlorides and vinyl fluorides, fluorinated alkenes such as perfluoroalkenes, e.g., perfluorodecene, acrylonitrile, methacrylonitrile, ethylene, propylene, allyl amine, vinylidene halides, butadienes, acrylamide, such as N-isopropylacrylamide and methacrylamide), epoxy compounds (including, e.g., glycidoxypropyltrimethoxysilane, glycidol, styrene oxide, butadiene monoxide, ethyleneglycol diglycidylether, and glycidyl methacrylate), and combinations thereof.

In at least one example, the organic compound comprises at least one phosphorylcholine group. For example, the organic compound may comprise a hydrocarbon monomer with at least one phosphorylcholine group, e.g., the coating comprising a polymer that includes one or more phosphorylcholine groups. Additionally or alternatively, the organic compound comprises a polymer formed from the polymerization of compounds that include one or more phosphorylcholine groups and a hydrocarbon monomer (including, e.g., acrylates and methacrylates, such as tert-butyl methacrylate and glycidyl methacrylate). The hydrocarbon monomer may be polar or non-polar. Exemplary polymers with one or more phosphorylcholine groups suitable for the coatings herein include, but are not limited to, 2-methacryloyloxyethyl-phosphorylcholine, phosphorylcholine glycol methacrylate, polyphosphorylcholine glycol acrylate, 2-methacryloyloxyethyl phosphorylcholine-co-n-butyl methacrylate, 2-hydroxy-3-methacryloxypropyltrimethylammonium chloride-2-methacryloxyethyl phosphorylcholine copolymer, and polyquaternium-64.

As mentioned above, the organic compounds(s) may be dissolved in a suitable solvent to form a liquid, which then may be used to generate an aerosol introduced into the plasma. Optionally, the organic compounds(s) may be dissolved in the solvent for at least 24 hours prior to introducing the aerosol into the non-thermal plasma, such as at least 36 hours or at least 48 hours before introducing the aerosol into the non-thermal plasma. The solvent may be selected based on chemical properties of the organic compound, e.g., a hydrophilic solvent for a hydrophilic organic compound, a hydrophobic solvent for a hydrophobic organic compound, etc. Exemplary solvents useful for generating the aerosol include, but are not limited to, water and aqueous solutions, alcohols, and volatile hydrocarbon liquids such as pentane, hexane, heptane, toluene, etc. A nebulizer may be used to generate an aerosol from the solution comprising the organic compound(s) dissolved in the solvent. The aerosol introduced into the non-thermal plasma may consist of or consist essentially of the organic compound (e.g., pharmaceutical agent or biomolecule) and the solvent, e.g., the aerosol does not include a binder, linker, or crosslinker.

The plasma may be generated using a non-thermal plasma device. The device can be used to deposit the coating and may also help to activate the substrate surface during deposition of the coating. The substrate may be exposed to the plasma before and/or during the deposition step. The plasma may be powered at a frequency of at least 10 KHz, such as greater than 20 KHz or greater than 125 KHz. The maximum frequency may be less than 1 MHZ, such as less than 900 kHz or less than 750 KHz. In at least one example, the plasma is operated at a frequency between 10 KHz and 99 kHz, for example between 15 KHz and 65 KHz.

In at least one aspect, the plasma is a pulsed plasma. The plasma may be pulsed at various duty cycles such that the power delivered is less than 100 W, such as less than 20 W or less than 10 W. The pulsing may be such that the applied power is off for at least 50% of the time and with the pulses switched on and off many times per second. For example, the plasma may be pulsed on and off to deliver an on time of 1 to 500 milliseconds, such as 10 to 300 milliseconds. For certain embodiments, particularly those involved with the treatment of tissue, especially cancer containing tissue, the plasma may be a nano-second or pico-second pulsed plasma. In these configurations, the plasma is only turned on for fractions of a millisecond for each pulse, for example less than 500 nano-seconds or less than 100 nano-seconds.

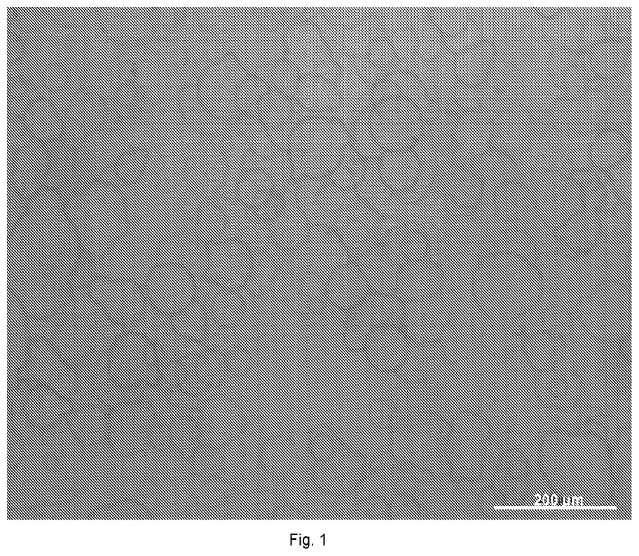

The plasma may then induce subsequent reactivity such that the solvent of the aerosol evaporates, and the organic compound(s) (e.g., polymer formed from a monomer, pharmaceutical agent, and/or biomolecule) forms a dry coating upon impacting a substrate and adheres to the substrate surface. The plasma may also activate the substrate surface, e.g., to increase the adhesion of the coating. The coating may be at least partially non-uniform and/or discontinuous, that is, the coating may include one or more defects. Exemplary defects may include surface roughness, a scratch, a crack, a ridge, a depression, density variation, phase change, wrinkling, chipping, blistering, bubbling, running, staining, water marking, streaking, hazing, uneven coating, and/or pinhole formation. For example, the coating may include a number of circular artifacts (see FIG. 1 discussed below). The edges of these circles (which may include ridges and/or depressions, and/or inhomogeneities like density variations, wrinkling, etc.) may act as nucleation points that allow cells (e.g., mammalian and bacterial cells) to adhere to the coated substrate. This may have several disadvantages, including reducing efficacy of the coating and/or limiting the ability of the coating to serve as a non-adherent surface.

After the coating is deposited, a solvent may be sprayed on the coating to increase uniformity of the coating. For example, at least part of defect(s), e.g., non-uniformity, may be decreased or eliminated (removed) by spraying a fine mist of solvent (e.g., a fine aerosol) onto the surface of the coated substrate. For example, spraying the solvent on the coating may help to smooth out the surface and promote greater uniformity (e.g., greater uniformity in the thickness and/or density) of the coating. The solvent may be sprayed on the coating without introducing the solvent into the non-thermal plasma, e.g., in the absence of a plasma. The solvent may be sprayed on the coating while the coating is dry and/or while the coating is at least partially wet. The solvent may comprise any suitable liquid such as water, methanol, ethanol, propanol, butanol, acetic acid, acetone, acetonitrile, butanone, carbon tetrachloride, chlorobenzene, chloroform, chlorohexane, diglyme, dimethylsulphoxide, methylene chloride, ethyl acetate, pentane, hexane, heptane, toluene, tetrahydrofuran, triethylamine, N,N-dimethylacetimide, cyclohexanone, and xylene or mixtures thereof. Solvents that are at least partially hydrophilic, e.g., alcohols such as ethanol, propanol, and methanol, may be useful for coatings comprising organic compounds that are at least partially hydrophilic. Such solvents are generally volatile and may be readily evaporated from the surface of the coating, such as via gas flow from a pneumatic nebulizer or similar device. Use of a nebulizer or similar device may eliminate the need for a subsequent drying step.

The same or different solvents may be used in the aerosol and treating the coating after deposition to enhance uniformity. Each solvent may be selected based on compatibility with the organic compound, e.g., having a similar hydrophobicity or hydrophilicity. In at least one example, the organic compound comprises a perfluorocarbon such as heptadecafluorodecyl acrylate or perfluorodecene, and the solvent used to treat the coating comprises cyclohexanone, N,N-dimethylacetimide, or a perfluorinated solvent such as perfluorohexane.

Surprisingly, it has been found that the uniformity of the coating may be improved if the organic compound is dissolved in the desired solvent and then stored for a few days before being provided to the substrate, e.g., via a plasma process. For example, certain organic compounds such as phosphorylcholine may be more fully dissolved after a few days in solution. Once the organic compound is added to the solvent, for example, the solution may be allowed to sit for at least 12 hours and up to 5 days or more, e.g., about 24 hours to about 3 days, or about 2 days to 3 days, before being provided to the substrate.

Surprisingly, it has also been found that adding a small amount (e.g., less than 50% by volume, or less than 30% by volume, such as about 1% to about 50% by volume, or about 5% to about 25% by volume) of an aqueous acid solution, e.g., an organic acid such as acetic acid, to the dissolved organic compound in solvent may promote uniformity. In at least one example, the organic compound comprises phosphorylcholine, which is dissolved in ethanol and less than 50% by volume of acetic acid is added. Optionally, the phosphorylcholine solution is allowed to sit for about 1 day to about 4 days before providing the solution to the substrate.

The volume of solvent used in the solvent spraying step may be controlled to provide the desired increased uniformity without deterioration of the coating (e.g., via excessive dissolution of deposited organic compound(s). The amount of fluid provided to the coating may range from about 0.1 L/cm² to about 1 ml/cm², such as about 0.1 μL/cm² to about 500 μL/cm², about 0.1 μL/cm² to about 100 μL/cm², about 0.1 μL/cm² to about 50 μL/cm², about 0.1 μL/cm² to about 10 μL/cm², about 5 μL/cm² to about 300 μL/cm², about 25 μL/cm² to about 500 μL/cm², about 100 μL/cm² to about 500 μL/cm², about 50 μL/cm² to about 100 μL/cm², about 250 μL/cm² to about 1 ml/cm², about 500 μL/cm² to about 750 μL/cm², or about 0.1 ml/cm² to about 1 ml/cm². In at least one example, the solvent spraying step results in about 0.2 ml/cm² to about 0.4 ml/cm² provided to the coating. Optionally, multiple passes may be carried out to achieve the total volume desired. Solvents that are highly volatile may lead to quicker drying. Further, a relatively lower flow rate may avoid, inhibit, or prevent excessive wetting of the coating, facilitating prompt drying of the coating. In such cases, the coating may be sufficiently dry shortly or immediately after providing the fluid so as to eliminate a subsequent drying step. In some examples, the amount of fluid provided to the coating may be less than or equal to 0.5 ml/cm² or less than or equal to 10 μL/cm², e.g., to prevent excessive wetting of the surface. The solvent may be sprayed over the surface for a time of 5 minutes or less, 3 minutes or less, 2 minutes or less, or 1 minute or less, e.g., about 30 seconds or about 15 seconds, to apply the desired volume per unit area of the coating surface. For example, the solvent may be sprayed over the surface for a time from about 15 seconds to about 5 minutes, from about 20 seconds to about 3 minutes, from about 30 seconds to about 1 minute, from about 20 seconds to about 45 seconds, from about 30 seconds to about 1.5 minutes, from about 1 minute to about 2 minutes, from about 30 seconds to about 5 minutes, from about 3 minutes to about 5 minutes, from about 1 minute to about 3.5 minutes, or from about 20 seconds to about 2 minutes.

Exemplary substrates that can be coated according to the methods herein include, but are not limited to, glass, plastics (such as, e.g., polystyrene, cyclic olefin copolymer, cyclic olefin polymer, polypropylene, and polycarbonate), fabrics and other textiles, silicones, ceramics, composite materials, and metals and metal alloys (e.g., Nitinol, titanium, stainless steel, etc.). The coatings disclosed herein may be applied to a medical device, e.g., to modify the surface of the medical device. Examples of medical devices suitable for the present disclosure include, but are not limited to, scalpels, clamps, needles, microplates, petri dishes, cell culture flasks, tubes, cuvettes, and other medical implants such as stents, shunts, drainage devices, catheters, ports, expandable balloons, prosthetic implants, orthopedic implants, dental implants, cochlear implants, car tubes, implantable mesh, spinal cages, maxillofacial implants, scaffolding (e.g., for tissue regeneration or grafting), pulse generators, valves, hormone delivery implants, skin grafts, bone grafts, artificial eye lenses, contact lens, hearing aids, breast implants, trauma fixation devices, screws, plates, rods, pins, nails, needles, biosensors, sensory implants, neural implants, pacemakers, defibrillators, electrodes, subcutaneous implants including drug delivery implants, cosmetic implants, hip implants and knee replacement implants, blood dialysis equipment, blood contacting tubing and bags, ventilators and associated tubes, valves and other components in contact with blood. In at least one example, the substrate is or comprises a microplate, a petri dish, a cell culture flask, a tube, or a cuvette.

In some aspects of the present disclosure, the substrate may be pretreated, e.g., via one or more pretreatments steps. Pretreatment processes suitable for the methods herein include, for example, plasma activation (under reduced pressure/vacuum or at atmospheric pressure), flame treatment, and chemical oxidation. Without being bound by theory, it is believed that the pretreatment step may activate the surface and/or prepare the surface for activation during application of the coating, which may enhance the uniformity of the coating. For example, pretreating the substrate surface may promote even spread and flow of a partially wet coating material before it cures completely, facilitating the creation of a more uniform deposition.

The coatings herein may have a thickness ranging from about 1 nm to about 500 nm, e.g., about 10 nm to about 100 nm, about 20 nm to about 50 nm, about 75 nm to about 300 nm, about 100 nm to about 450 nm, about 150 nm to about 250 nm, about 35 nm to about 85 nm. In at least one example, the coating has a thickness of less than 100 nm and greater than 10 nm. According to some aspects of the present disclosure, the coating has a thickness less than or equal to 500 nm, such as a thickness ranging from about 20 nm to about 50 nm.

Using the methods herein, it has been possible to produce a range of coatings that adhere to the substrate and have enhanced uniformity. As mentioned above, coatings can contain a number of defects or irregularities that act as nucleation points that can allow cells (e.g., mammalian and bacterial cells) to adhere to the surface of the coating. This can reduce the efficacy of the coating, particularly in biological environments, and thus can limit the viability of the coating to provide a non-adherent surface. However, when the coating is subsequently exposed to a suitable solvent (which may be applied as a fine mist), these defects or irregularities may be substantially or completely removed.

Without being bound by theory, it is believed that the solvent may at least partially dissolve the outermost surface of the coating (e.g., dissolving an outermost layer or partial layer of organic compound(s)), which allows the deposited materials to undergo physical re-arrangements that can alter the chemical and/or physical properties of the coating. For example, the deposited organic material(s) (the organic compound(s) or a polymer formed from the organic compound(s)) can undergo self-assembly reactions to form organized structures on the surface. Once dissolved in the solvent, the organic molecules may be temporarily free to move. The molecules may rearrange via increased degrees of freedom provided by the solvent fluid to increase uniformity and removing or otherwise minimizing defects in the coating that could act as attachment points for cells or other materials. Without the solvent spraying step, such rearrangement may be hindered, e.g., by cross-linking induced by the plasma.

In some aspects of the present disclosure, the solvent spraying step may be used to deposit further material on the substrate surface, e.g., a further coating comprising the material and/or to incorporate the material into the coating (e.g., providing a thicker coating) deposited via the non-thermal plasma. For example, the solvent may comprise one or more other materials in addition to the solvent, wherein the one or more other materials may be dissolved in the solvent. The further material(s) may be the same or different than the organic compound and may be applied with the solvent in the absence of plasma. Without being bound by theory, it is believed that, in some cases, the initial non-thermal plasma deposition may produce a coating that is well adhered but that may contain non-uniformities. By spraying the material and solvent (e.g., as a subsequent layer of organic compound or other coating material and solvent) but with no plasma present, a layer may be applied that dries relatively slowly and produces a more uniform coating and surface finish to the coating. This second layer may also partially dissolve the uppermost part of the plasma-deposited layer and dry out to produce a single, thicker layer which may be previously adhered to the substrate, e.g., via the non-thermal plasma process. In this way, the solvent spraying step may retain the uniformity of a wet spray process. In some examples, the volume of solvent and material(s) added via the solvent spraying step in absence of plasma does not exceed the total solvent volume added in the first deposition step using the non-thermal plasma (that is, the total volume of aerosol introduced into the non-thermal plasma and subsequently deposited on the substrate surface). In this way, limiting the amount of material(s) added in the solvent spraying step may avoid excessive or complete dissolution of the plasma coating from the substrate.

Coated substrates can have various applications, including industrial and medical, for example. Coated substrates according to the present disclosure may be useful as non-fouling surfaces, antimicrobial devices, and cell culture processes. Coated substrates according to the present disclosure can also be used, for example, to inhibit blood clotting on implant or other medical device surface or blood contacting materials. The organic compound(s), e.g., active agent(s), of the coating may provide the coating with an ability to inhibit blood clotting, which may be useful for medical implants and other medical device surfaces or blood contacting materials. The coatings prepared according to the methods herein may be optically clear, e.g., transparent.

The organic compounds of the coating may be useful to inhibit or prevent adhesion of materials to the coating. For example, the coatings prepared according to the methods herein may provide a hydrophobic surface capable of repelling cells and preventing cell adhesion. Some compounds, such as phosphorylcholine materials, have been known to suffer from poor adhesion to the substrate that can result in delamination. The methods herein avoid such problems, e.g., via increased adhesion provided by the plasma deposition process. When deposited using the methods herein, the coating may exhibit excellent adhesion to the substrate, e.g., the coating is not easily removed from the substrate.

Substrates with coating comprising organic compounds with phosphorylcholine groups may be useful to alter the adhesion of cells (e.g., mammalian cells), microbes, and/or other materials. Coatings comprising phosphorylcholine groups may exhibit water stability and long-term biocompatibility, while also retaining biological functionality of the phosphorylcholine moiety(ies). After the spraying step, the coatings herein with increase uniformity may be capable of inhibiting adhesion of bacterial and/or eukaryotic cells while maintaining strong adherence of the coating to the substrate. Such coatings may provide antimicrobial protection for medical devices.

The following examples are intended to illustrate the present disclosure without, however, being limiting in nature. It is understood that the present disclosure encompasses additional embodiments consistent with the foregoing description and following examples.

### EXAMPLES

### Example 1

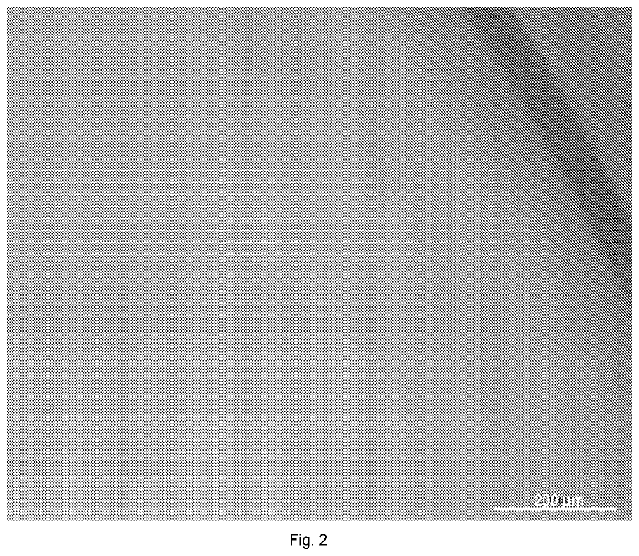

FIGS. 1 and 2 illustrate coating deposited on a polystyrene substrate before (FIG. 1) and after (FIG. 2) an aerosol spraying step using ethanol as the solvent. The coating was deposited by dissolving a phosphorylcholine polymer (Lipidure®, NOF Corp.) in ethanol to give a concentration of 8 mg/ml, and then spraying the liquid as an aerosol into a non-thermal helium plasma operating at 20 kHz. The substrate was positioned about 2-5 mm downstream of the afterglow region of the plasma. The phosphorylcholine polymer deposited on the substrate to form a coating about 20-80 nm thick. For the spraying step following the image captured as FIG. 1 (using an optical microscope), ethanol was used as the solvent. A total volume of about 0.004 ml/cm² was sprayed uniformly over the surface over a time period of about 2 minutes. As evident in FIG. 2, the spraying step resulting in removal of circular artifacts/irregularities initially present in the coating. The size bars in FIGS. 1 and 2 are 200 μm.

### Example 2

A phosphorylcholine polymer (Lipidure®, NOF Corp.) was dissolved in ethanol to give a solution of 8 mg/ml. This solution was allowed to dissolve over a period of about 3 days and then diluted 2:1 in acetic acid to give a final concentration of 5.3 mg/ml. The diluted solution was deposited onto a polystyrene microplate by spaying the solution as an aerosol at a flow rate of 100 μL/min and at a speed of 1 m/s into a cold (non-thermal) helium plasma operating at 20 KHz, the microplate being positioned about 2-5 mm downstream of the afterglow region of the plasma. This method was repeated three times over a period of 6 minutes to produce a coating with 3 layers. The coating was about 30-100 nm thick.

After coating, some minor imperfections could be seen and the surface was slightly more hydrophilic than the initial polystyrene. Therefore, the surface was sprayed with a nebulized flow of ethanol comprising water (2:1 ethanol: water) at a flow rate of 200 μL/min. The spray coating took 4 minutes and 21 seconds, which equates to a dose of about 7.9 μL/cm² and was immediately dry upon completion of the spraying process. No additional heating or drying steps were performed. Following this treatment, the coating was optically clear (transparent) and had a hydrophobic surface that was capable of repelling cells and preventing cell adhesion.

### Example 3

A thin coating layer was deposited on top of an adherent plasma-deposited coating. A phosphorylcholine polymer (Lipidure®, NOF Corp.) was dissolved in ethanol to give a concentration of 8 mg/ml. This solution was allowed to sit for 3 days to promote complete dissolution, and then deposited onto a titanium substrate surface by spaying the solution as an aerosol into a non-thermal helium plasma using a flow rate of 200 μL/min and a speed of 1 m/min (˜16.7 mm/s). The non-thermal plasma was operated at 20 kHz. This produced a well-adhered coating but with a certain degree of roughness apparent by visual inspection. In order to smooth this out, a subsequent layer of the phosphorylcholine polymer/ethanol solution was sprayed on the substrate surface using the same conditions (flow rate of 200 μL/min and a speed of 1 m/min) but with the helium plasma turned off. This second layer was allowed to dry in air and then examined. The coating was found to have a smoother and more uniform finish while also adhering to the plasma-deposited base layer to form a highly uniform and adherent coating. The coating was about 60 nm thick. Without being bound by theory, it is believed that the plasma-deposited layer acted as a primer that allowed for subsequent layers of phosphorylcholine polymer to bind strongly to the surface and resist delamination. A further level of smoothening was obtained by spraying a layer of ethanol at 200 to 400 μL/min, which produced a highly uniform finish that was resistant to cell adhesion.

### Example 4

A phosphorylcholine polymer (Lipidure®, NOF Corp.) was dissolved in ethanol to give a concentration of 8 mg/mL. Water and additional ethanol were then added to the solution to give a concentration of 1.6 mg/mL phosphorylcholine and a 1:1 ratio of water to ethanol. The solution was then allowed to age to promote complete dissolution for 2 days. In the meantime, Nitinol substrates were pretreated in a vacuum plasma system (a helium plasma operating at 13.56 MHz and pressure of 0.3 mbar) and then transferred to a non-thermal helium atmospheric pressure plasma coating system as described in Examples 1-3.

The phosphorylcholine polymer solution was deposited onto the pretreated Nitinol substrate surfaces by spaying the solution as an aerosol (nebulized using a T2100 nebulizer) into a non-thermal helium plasma at a liquid flow rate of 100 μL/min and a speed of 1 m/min (˜16.7 mm/s). The plasma was passed over each substrate surface 4 to 12 times to give coatings of varying thicknesses (each substrate having a thickness between 20 nm and 80 nm). The resultant coatings were found to be highly uniform.

To ensure uniformity, a solvent spraying step was performed without the use of plasma power, spraying a solvent comprising water and ethanol at a 1:1 ratio of water:ethanol at a flow rate of 100 μL/min, and a speed of 1 m/min (˜16.7 mm/s) to each coated substrate. This method produced surfaces that were highly uniform when examined under an optical microscope.

It is intended that the specification and examples therein be considered as exemplary only. Other aspects and embodiments of the present disclosure will be apparent to those skilled in the art from consideration of the specification and claims, and practice of the embodiments disclosed herein.

What is claimed is:

1. A method for preparing a substrate, the method comprising:
introducing an aerosol into a non-thermal plasma, wherein the aerosol comprises at least one organic compound and a first solvent:
depositing the at least one organic compound on a surface of a substrate positioned downstream of the plasma to form a coating on the surface, and
spraying a second solvent in aerosol form on the coating, the second solvent being the same or different from the first solvent:
wherein the spraying the second solvent increases uniformity of the coating.

2. The method of claim 1, wherein the second solvent is sprayed on the coating within 1 hour to 24 hours of forming the coating by depositing the at least one organic compound on the surface of the substrate.

3. The method of claim 1 or 2, wherein the second solvent comprises an alcohol such as ethanol, methanol, propanol, or butanol, or wherein the second solvent comprises N,N-dimethylacetimide or cyclohexanone.

4. The method of any one of the preceding claims, wherein the at least one organic compound comprises a pharmaceutical agent or a biomolecule, and the coating retains a pharmaceutical activity or biological activity of the at least one organic compound.

5. The method of any one of the preceding claims, wherein the organic compound comprises at least one phosphorylcholine group.

6. The method of any one of the preceding claims, wherein less than or equal to 1 ml/cm² of the second solvent is sprayed on the coating, such as less than or equal to 100 μL/cm² of the second solvent.

7. The method of any one of the preceding claims, wherein less than or equal to 10 μL/cm² of the second solvent is sprayed on the coating.

8. The method of any one of the preceding claims, wherein the coating has a thickness less than or equal to 500 nm, such as about 20 nm to about 50 nm.

9. The method of any one of the preceding claims, wherein the substrate comprises glass, a plastic, a metal, or a metal alloy.

10. The method of any one of the preceding claims, wherein the substrate comprises a medical device, optionally wherein the medical device is a microplate, a petri dish, a cell culture flask, a tube, or a cuvette.

11. The method of any one of the preceding claims, wherein the at least one organic compound is dissolved in the first solvent for at least 48 hours prior to introducing the aerosol into the non-thermal plasma.

12. The method of any one of the preceding claims, further comprising preparing a solution comprising the at least one organic compound and the first solvent, and introducing the solution into a nebulizer to form the aerosol at least 48 hours after preparing the solution.

13. The method of any one of the preceding claims, wherein the second solvent is not introduced into the non-thermal plasma before spraying the second solvent on the coating.

14. The method of any one of the preceding claims, further comprising pretreating the surface of the substrate before depositing the at least one organic compound on the surface, optionally wherein pretreating the surface includes plasma activation at reduced pressure, plasma activation at atmospheric pressure, flame treatment, or chemical oxidation.

15. The method of any one of the preceding claims, wherein the organic compound is a first organic compound, and the second solvent is sprayed on the coating as an aerosol that comprises the second solvent and a second organic compound that is the same or different than the first organic compound.

16. The method of any one of the preceding claims, wherein the second solvent is sprayed on the coating while the coating is wet.

17. The method of any one of claims 1-15, wherein the second solvent is sprayed on the coating while the coating is dry.

18. The method of any one of the preceding claims, wherein the coating includes one or more defects, and spraying the second solvent decreases or eliminates the one or more defects.

19. The method of any one of the preceding claims, wherein the one or more defects includes surface roughness, a scratch, a crack, a ridge, and/or a depression.

20. The method of any one of the preceding claims, wherein a total volume of the second solvent applied to the substrate surface does not exceed a total volume of the first solvent applied to the substrate surface.

21. The method of any one of the preceding claims, wherein the aerosol introduced into the non-thermal plasma consists of or consists essentially of a pharmaceutical agent and the first solvent, or wherein the aerosol introduced into the non-thermal plasma consists of or consists essentially of a biomolecule and the first solvent.

22. A substrate prepared according to the method of any one of the preceding claims, wherein the substrate is a medical device, and wherein the coating is a uniform coating having a thickness less than or equal to 500 nm, such as about 20 nm to about 50 nm.

23. The substrate of claim 22, wherein the medical device is a microplate, a petri dish, a cell culture flask, a tube, or a cuvette.

24. The substrate of claim 22 or 23, wherein the coating comprises phosphorylcholine.

25. The substrate of any one of claims 22-24, wherein the coating inhibits or prevents cell adhesion to the substrate.
